SEQUENCE LISTING (53 sequences)

SEQ ID NO: 1 - DNA, 156 bp, Artificial Sequence
  Synthetic oligonucleotide
ccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactc

SEQ ID NO: 2 - DNA, 29 bp, Artificial Sequence
  Synthetic oligonucleotide
agagaaacattcaggacctcatcattatg

SEQ ID NO: 3 - DNA, 19 bp, Artificial Sequence
  Synthetic oligonucleotide
cagguccttcttgcctccc

SEQ ID NO: 4 - DNA, 19 bp, Artificial Sequence
  Synthetic oligonucleotide
gcagccaagaaggccatct

SEQ ID NO: 5 - DNA, 46 bp, Artificial Sequence
  Synthetic oligonucleotide
aatttaatacgactcactatagggagagcagccaagaaggccatct

SEQ ID NO: 6 - DNA, 22 bp, Artificial Sequence
  Synthetic oligonucleotide
tatggaggctccaattgaaacc

SEQ ID NO: 7 - DNA, 49 bp, Artificial Sequence
  Synthetic oligonucleotide
aatttaatacgactcactatagggagatatggaggctccaattgaaacc

SEQ ID NO: 8 - DNA, 22 bp, Artificial Sequence
  Synthetic oligonucleotide
gctttgttctccacagggtcag

SEQ ID NO: 9 - DNA, 18 bp, Artificial Sequence
  Synthetic oligonucleotide
ctttgttctccacagggt

SEQ ID NO: 10 - DNA, 27 bp, Artificial Sequence
  Synthetic oligonucleotide
ctgtcttttatttctagccccttttgg

SEQ ID NO: 11 - DNA, 31 bp, Artificial Sequence
  Synthetic oligonucleotide
gtcttttatttctagccccttttggaacagg

SEQ ID NO: 12 - DNA, 24 bp, Artificial Sequence
  Synthetic oligonucleotide
gggctggtgaatgcacgctgatgg

SEQ ID NO: 13 - DNA, 26 bp, Artificial Sequence
  Synthetic oligonucleotide
guggcgatgggctggtgaatgcacgc

SEQ ID NO: 14 - DNA, 28 bp, Artificial Sequence
  Synthetic oligonucleotide
gaguttgtggcgatgggctggtgaatgc

SEQ ID NO: 15 - DNA, 23 bp, Artificial Sequence
  Synthetic oligonucleotide
caccaactgggggtatatacccc

SEQ ID NO: 16 - DNA, 62 bp, Artificial Sequence
  Synthetic oligonucleotide
aatttaatacgactcactatagggagaccacaacggtttcaccaactgggggtatatacccc

SEQ ID NO: 17 - DNA, 25 bp, Artificial Sequence
  Synthetic oligonucleotide
gggggtatataccccaacactaggc

SEQ ID NO: 18 - DNA, 64 bp, Artificial Sequence
  Synthetic oligonucleotide
aatttaatacgactcactatagggagaccacaacggtttgggggtatataccccaacactaggc

SEQ ID NO: 19 - DNA, 27 bp, Artificial Sequence
  Synthetic oligonucleotide
ggtatataccccaacactaggctcccc

SEQ ID NO: 20 - DNA, 66 bp, Artificial Sequence
  Synthetic oligonucleotide
aatttaatacgactcactatagggagaccacaacggtttggtatataccccaacactaggctcccc

SEQ ID NO: 21 - DNA, 25 bp, Artificial Sequence
  Synthetic oligonucleotide
ctccccaccagccatatgccttctc

SEQ ID NO: 22 - DNA, 64 bp, Artificial Sequence
  Synthetic oligonucleotide
aatttaatacgactcactatagggagaccacaacggtttctccccaccagccatatgccttctc

SEQ ID NO: 23 - RNA, 21 nt, Artificial Sequence
  Synthetic oligonucleotide
ucuuuaguaguaagugcccag

SEQ ID NO: 24 - RNA, 26 nt, Artificial Sequence
  Synthetic oligonucleotide
cugggucuuuaguaguaagugcccag

SEQ ID NO: 25 - RNA, 23 nt, Artificial Sequence
  Synthetic oligonucleotide
ccaggucuuuaguaguaagugcc

SEQ ID NO: 26 - RNA, 28 nt, Artificial Sequence
  Synthetic oligonucleotide
ggcucccaggucuuuaguaguaagugcc

SEQ ID NO: 27 - RNA, 19 nt, Artificial Sequence
  Synthetic oligonucleotide
ccaggucuuuaguaguaag

SEQ ID NO: 28 - RNA, 24 nt, Artificial Sequence
  Synthetic oligonucleotide
ccaggucuuuaguaguaagccugg

SEQ ID NO: 29 - RNA, 24 nt, Artificial Sequence
  Synthetic oligonucleotide
cuccgccaggucuuuaguaguaag

SEQ ID NO: 30 - RNA, 29 nt, Artificial Sequence
  Synthetic oligonucleotide
cuccgccaggucuuuaguaguaagcggag

SEQ ID NO: 31 - RNA, 25 nt, Artificial Sequence
  Synthetic oligonucleotide
cuccauuacgcuguguccuuucucc

SEQ ID NO: 32 - RNA, 28 nt, Artificial Sequence
  Synthetic oligonucleotide
cuuccccuuucuccauuacgcugugucc

SEQ ID NO: 33 - RNA, 34 nt, Artificial Sequence
  Synthetic oligonucleotide
gcgcauuuuuguuucugaauucuacuacuucccc

SEQ ID NO: 34 - DNA, 28 bp, Artificial Sequence
  Synthetic oligonucleotide
catttgacaaacaaagaaagagatgcgc

SEQ ID NO: 35 - DNA, 29 bp, Artificial Sequence
  Synthetic oligonucleotide
cagacaattccagttaaaattttcatttg

SEQ ID NO: 36 - RNA, 27 nt, Artificial Sequence
  Synthetic oligonucleotide
ccaaacauccuauuuccuuggcucucc

SEQ ID NO: 37 - RNA, 24 nt, Artificial Sequence
  Synthetic oligonucleotide
gagaggcugacgccauuugggugc

SEQ ID NO: 38 - RNA, 24 nt, Artificial Sequence
  Synthetic oligonucleotide
ccuauuuccuuggcucucccuugc

SEQ ID NO: 39 - RNA, 25 nt, Artificial Sequence
  Synthetic oligonucleotide
uaacacuggguuugguauaacacug

SEQ ID NO: 40 - RNA, 23 nt, Artificial Sequence
  Synthetic oligonucleotide
cugaauucuacuacuuccccuuu

SEQ ID NO: 41 - RNA, 34 nt, Artificial Sequence; identical to SEQ ID NO: 33
  Synthetic oligonucleotide

SEQ ID NO: 42 - RNA, 45 nt, Artificial Sequence
  Synthetic oligonucleotide
cagacaauuccaguuaaaauuuucauuugacaaacaaagaaagag

SEQ ID NO: 43 - DNA, 35 bp, Artificial Sequence
  Synthetic oligonucleotide
ccgcctacccaaaatgcctgcgtgatttctgattg

SEQ ID NO: 44 - RNA, 25 nt, Artificial Sequence
  Synthetic oligonucleotide
cuggaggccgccuacccaaaaugcc

SEQ ID NO: 45 - RNA, 27 nt, Artificial Sequence
  Synthetic oligonucleotide
cgacucaaaggaaaacuggaggccgcc

SEQ ID NO: 46 - DNA, 27 bp, Artificial Sequence
  Synthetic oligonucleotide
aatttaatacgactcactatagggaga

SEQ ID NO: 47 - DNA, 3112 bp, Artificial Sequence
  Synthetic oligonucleotide
gggaacaaaagctggagctccaccgcggtggcggccgctctagccctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagaaccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttgaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcgggcggatcccccgggctgcaggaattcgatatcaagcttatcgataccgtcga

SEQ ID NO: 48 - RNA, 25 nt, Artificial Sequence
  Synthetic oligonucleotide
ggugaauuccaguauggguuugggg

SEQ ID NO: 49 - RNA, 27 nt, Artificial Sequence
  Synthetic oligonucleotide
cccccaguuggugaauuccaguauggg

SEQ ID NO: 50 - RNA, 27 nt, Artificial Sequence
  Synthetic oligonucleotide
gguauauacccccaguuggugaauucc

SEQ ID NO: 51 - RNA, 23 nt, Artificial Sequence
  Synthetic oligonucleotide
gggguauauacccccaguuggug

SEQ ID NO: 52 - RNA, 24 nt, Artificial Sequence
  Synthetic oligonucleotide
ccuaguguugggguauauaccccc

SEQ ID NO: 53 - RNA, 18 nt, Artificial Sequence
  Synthetic oligonucleotide
ggggagccuaguguuggg

(19) United States
(12) Patent Application Publication
(10) Pub. No.: US 2010/0196903 A1
(43) Pub. Date: Aug. 5, 2010

(54) COMPOSITIONS AND METHODS TO DETECT TMPRSS2/ERG TRANSCRIPT VARIANTS IN PROSTATE CANCER

(75) Inventors: Paul M. DARBY, San Diego, CA (US); Siobhan M. MIICK, San Diego, CA (US); Kumar KASTURY, San Jose, CA (US); Jo Ann JACKSON, Lakeside, CA (US)

Correspondence Address: GEN PROBE INCORPORATED, 10210 GENETIC CENTER DRIVE, Mail Stop #1 / Patent Dept., SAN DIEGO, CA, 92121, US

(73) Assignee: GEN-PROBE INCORPORATED, San Diego, CA (US)

(21) Appl. No.: 12/689,178

(22) Filed: Jan. 18, 2010

Related U.S. Application Data
(63) Continuation of application No. PCT/US08/70334, filed on Jul. 17, 2008.
(60) Provisional application No. 60/950,390, filed on Jul. 18, 2007.

(51) Int. Cl. C12Q 1/68

(52) U.S. Cl. 435/6

(57) ABSTRACT

Compositions and methods for detecting TMPRSS2/ERG transcript variants in prostate cancer are provided. The compositions and methods have utility in prostate cancer diagnosis.

### RELATED APPLICATIONS

This application claims the benefit under 35 U.S.C. §§119(e) and 363 to application no. PCT/US08/70334, filed Jul. 17, 2008, which claims the benefit under 35 U.S.C. §119(e) of provisional application No. 60/950,390, filed Jul. 18, 2007, which are incorporated by reference herein.

### FIELD OF THE INVENTION

The present invention relates to compositions and methods for cancer diagnosis and research, including but not limited to, cancer markers. In particular, the present invention relates to recurrent gene fusions as diagnostic markers for prostate cancer.

### BACKGROUND OF THE INVENTION

A central aim in cancer research is to identify altered genes that are causally implicated in oncogenesis. Several types of somatic mutations have been identified including base substitutions, insertions, deletions, translocations, and chromosomal gains and losses, all of which result in altered activity of an oncogene or tumor suppressor gene. First hypothesized in the early 1900's, there is now compelling evidence for a causal role for chromosomal rearrangements in cancer (Rowley, Nat Rev Cancer 1: 245 (2001)). Recurrent chromosomal aberrations were thought to be primarily characteristic of leukemias, lymphomas, and sarcomas. Epithelial tumors (carcinomas), which are much more common and contribute to a relatively large fraction of the morbidity and mortality associated with human cancer, comprise less than 1% of the known, disease-specific chromosomal rearrangements (Mitelman, Mutat Res 462: 247 (2000)). While hematological malignancies are often characterized by balanced, disease-specific chromosomal rearrangements, most solid tumors have a plethora of non-specific chromosomal aberrations. It is thought that the karyotypic complexity of solid tumors is due to secondary alterations acquired through cancer evolution or progression.

Two primary mechanisms of chromosomal rearrangements have been described. In one mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (Rabbitts, Nature 372: 143 (1994)). In the second mechanism, rearrangement results in the fusion of two genes, which produces a fusion protein that may have a new function or altered activity. The prototypic example of this translocation is the BCR-ABL gene fusion in chronic myelogenous leukemia (CML) (Rowley, Nature 243: 290 (1973); de Klein et al., Nature 300: 765 (1982)). Importantly, this finding led to the rational development of imatinib mesylate (Gleevec®, manufactured by Novartis®), which successfully targets the BCR-ABL kinase (Deininger et al., Blood 105: 2640 (2005)). Thus, identifying recurrent gene rearrangements in common epithelial tumors may have profound implications for cancer drug discovery efforts as well as patient treatment.

### SUMMARY OF THE INVENTION

The present invention provides, but is not limited to, compositions and methods for amplifying and detecting TMPRSS2/ERG transcript variants.

A composition is provided that comprises a first amplification oligonucleotide comprising a sequence that specifically hybridizes to SEQ ID NO: 1, a second amplification oligonucleotide comprising a sequence that specifically hybridizes to SEQ ID NO: 1 and an oligonucleotide probe comprising a sequence that specifically hybridizes to SEQ ID NO: 1, such that the first and second amplification oligonucleotides specifically hybridize to different target sequences in SEQ ID NO: 1.

A method is provided for amplifying and detecting ERG transcripts in a biological sample comprising: contacting said sample containing ERG transcripts with a first amplification oligonucleotide that specifically hybridizes to SEQ ID NO: 1 and a second amplification oligonucleotide that specifically hybridizes to SEQ ID NO: 1, such that the first and second amplification oligonucleotides hybridize to different target sequences in SEQ ID NO: 1; exposing said sample contacted with said first and second amplification oligonucleotides to conditions that amplify ERG transcripts to make an amplified product; and detecting the presence of the amplified product by specifically hybridizing the product with a detection probe that specifically hybridizes to SEQ ID NO: 1 or a sequence completely complementary to SEQ ID NO: 1, thereby detecting the presence of ERG transcripts in the sample.

Another method is provided for amplifying and detecting TMPRSS2/ERG transcript variants in a patient sample comprising: contacting said patient sample with a first amplification oligonucleotide comprising a target specific sequence consisting of SEQ ID NO: 14, a second amplification oligonucleotide comprising a target specific sequence consisting of SEQ ID NO: 17 or 19, and a detection probe comprising a target specific sequence consisting of SEQ ID NO: 29; exposing said patient sample to conditions sufficient to amplify TMPRSS2/ERG transcript variants; and determining whether said TMPRSS2/ERG transcript variants are in said patient sample.

### DESCRIPTION OF THE FIGURES

FIG. 1 characterizes 12 different TMPRSS2/ERG transcript variants and the target region of the present invention.

FIG. 2 provides the polynucleotide sequence corresponding to SEQ ID NO: 47.

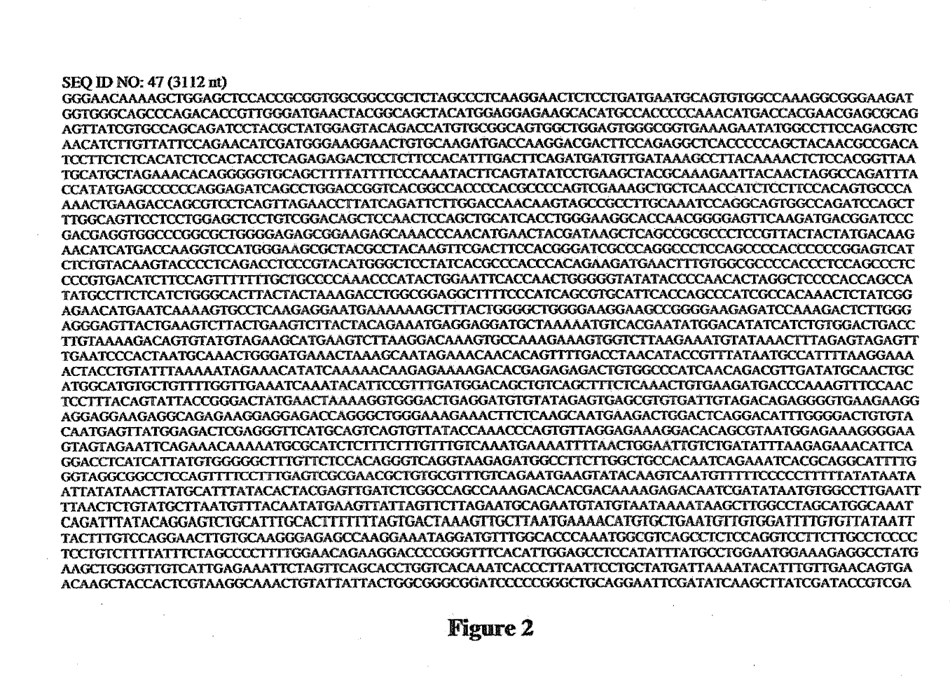

### DEFINITIONS

To facilitate an understanding of the present invention, a number of terms and phrases are defined below:

As used herein, the term “gene fusion” refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes.

As used herein, the term “transcriptional regulatory region” refers to the non-coding upstream regulatory sequence of a gene, also called the 5′ untranslated region (5′UTR).

As used herein, the term “androgen regulated gene” refers to a gene or portion of a gene whose expression is initiated or enhanced by an androgen (e.g., testosterone). The promoter region of an androgen regulated gene may contain an “androgen response element” that interacts with androgens or androgen signaling molecules (e.g., downstream signaling molecules).

As used herein, the terms “detect”, “detecting”, or “detection” may describe either the general act of discovering or discerning or the specific observation of a detectably labeled composition.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing prostate tissue in a subject” refers to the identification of one or more properties of a prostate tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “amplification oligonucleotide” refers to an oligonucleotide that hybridizes to a target nucleic acid, or its complement, and participates in a nucleic acid amplification reaction. An example of an amplification oligonucleotide is a “primer” that hybridizes to a template nucleic acid and contains a 3′ OH end that is extended by a polymerase in an amplification process. Another example of an amplification oligonucleotide is an oligonucleotide that is not extended by a polymerase (e.g., because it has a 3′ blocked end) but participates in or facilitates amplification. Amplification oligonucleotides may optionally include modified nucleotides or analogs, or additional nucleotides that participate in an amplification reaction but are not complementary to or contained in the target nucleic acid. Amplification oligonucleotides may contain a sequence that is not complementary to the target or template sequence. For example, the 5′ region of a primer may include a promoter sequence that is non-complementary to the target nucleic acid (referred to as a “promoter-primer”). Those skilled in the art will understand that an amplification oligonucleotide that functions as a primer may be modified to include a 5′ promoter sequence, and thus function as a promoter-primer. Similarly, a promoter-primer may be modified by removal of, or synthesis without, a promoter sequence and still function as a primer. A 3′ blocked amplification oligonucleotide may provide a promoter sequence and serve as a template for polymerization (referred to as a “promoter-provider”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE INVENTION

The present invention is based on the discovery of recurrent gene fusions in prostate cancer. The present invention provides diagnostic and research methods that either directly or indirectly detect the gene fusions. The present invention also provides compositions for diagnostic and research purposes.

### I. TMPRSS2/ERG Gene Fusions

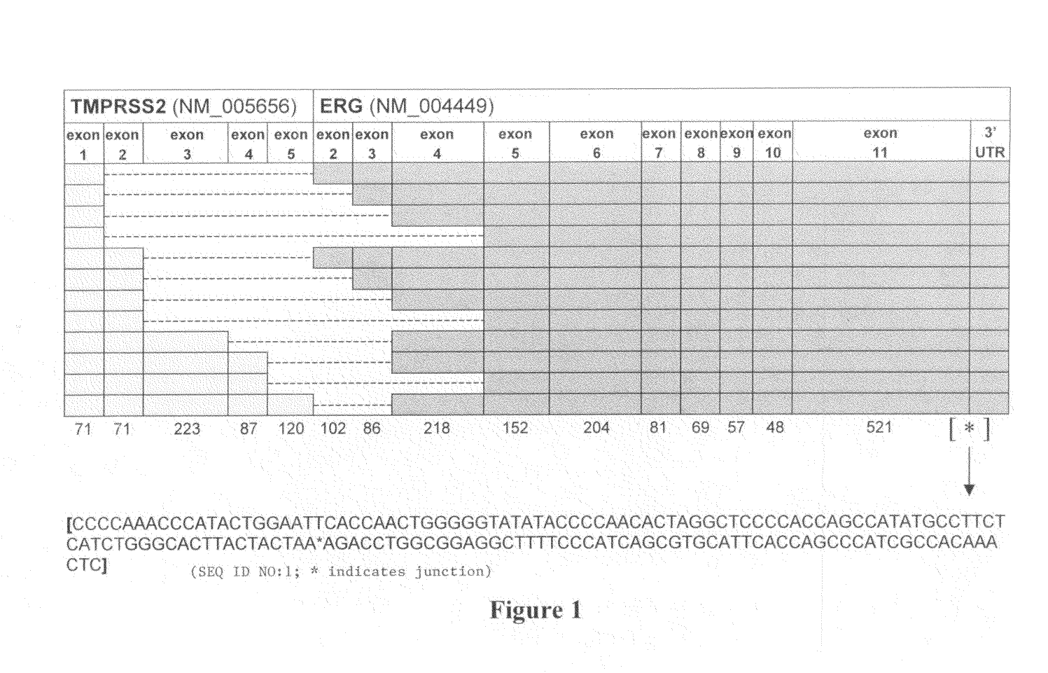

Recurrent gene fusions of the androgen regulated gene TMPRSS2 with ETS family member genes ERG, ETV1, ETV4, or FLI1 have recently been identified in 50-80% of prostate cancers (Int'l Publ. No. WO 2007/033187). Of those, 50-70% are attributable to chromosomal rearrangements fusing TMPRSS2 with ERG. Despite recurrence, the junction at which TMPRSS2 fuses to ERG varies. Consequently, at least 12 different TMPRSS2/ERG transcript variants have been described to date (Tomlins et al., Science 310: 644 (2005); Wang et al., Cancer Research 66(17): 8347 (2006); Soller et al., Genes Chromosomes Cancer 45(7): 717 (2006); Clark et al., Oncogene 26(18): 2667 (2007)). Characterization of the 12 different TMPRSS2/ERG transcript variants appears in FIG. 1. The present invention provides compositions and methods to detect multiple TMPRSS2/ERG transcript variants by targeting the region defined by SEQ ID NO: 1. Even though SEQ ID NO: 1 is given as DNA, the skilled artisan will appreciate that the corresponding RNA replaces all thymines (T) with uracil (U).

### II. Diagnostic Applications

The present invention provides DNA and RNA based diagnostic methods that either directly or indirectly detect the TMPRSS2/ERG transcript variants. The present invention also provides compositions and kits for diagnostic purposes.

The diagnostic methods of the present invention may be qualitative or quantitative. Quantitative diagnostic methods may be used, for example, to discriminate between indolent and aggressive cancers via a cutoff or threshold level. Where applicable, qualitative or quantitative diagnostic methods may also include amplification of target, signal or intermediary (e.g., a universal primer).

An initial assay may confirm the presence of TMPRSS2/ERG transcript variants but not identify the specific transcript variant. A secondary assay is then performed to determine the identity of the particular transcript variant, if desired. The second assay may use a different detection technology than the initial assay. The diagnostic methods of the present invention may also be modified with reference to data correlating a particular TMPRSS2/ERG transcript variant with the stage, aggressiveness or progression of the disease or the presence or risk of metastasis. Ultimately, the information provided by the methods of the present invention will assist a physician in choosing the best course of treatment for a particular patient.

The TMPRSS2/ERG transcript variants may be detected along with other markers in a multiplex or panel format. Markers are selected for their predictive value alone or in combination with the TMPRSS2/ERG transcript variants. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); and, those disclosed in U.S. Pat. Nos. 5,854,206 and 6,034,218, and U.S. Publication No. 20030175736. Markers for other cancers, diseases, infections, and metabolic conditions are also contemplated for inclusion in a multiplex of panel format.

A. Sample

Any patient sample suspected of containing the TMPRSS2/ERG transcript variants may be tested according to the methods of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

The patient sample typically requires preliminary processing designed to isolate or enrich the sample for the TMPRSS2/ERG transcript variants or cells that contain the TMPRSS2/ERG transcript variants. A variety of techniques known to those of ordinary skill in the art may be used for this purpose, including but not limited: centrifugation; immunocapture; cell lysis; and, nucleic acid target capture; all of which are described in EP Pat. No. 1 409 727.

B. DNA and RNA Detection

The TMPRSS2/ERG transcript variants may be detected using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

The TMPRSS2/ERG transcript variants may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (PCR) is described in detail in U.S. Pat. Nos. 4,683,195, 4,683,202, 4,800,159 and 4,965,188. Briefly, PCR uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988).

Transcription mediated amplification (TMA) is described in detail in U.S. Pat. Nos. 5,824,518, 5,480,784 and 5,399,491. Briefly, TMA synthesizes multiple copies of a target nucleic acid sequence autocatalytically under conditions of substantially constant temperature, ionic strength, and pH in which multiple RNA copies of the target sequence autocatalytically generate additional copies. In a variation described in U.S. Publ. No. 20060046265, TMA optionally incorporates the use of blocking moieties, terminating moieties, and other modifying moieties to improve TMA process sensitivity and accuracy.

The ligase chain reaction (LCR) is described in Weiss, R., Science 254: 1292 (1991). Briefly, LCR uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (SDA) is described in Walker, G. et al., Proc. Natl. Acad. Sci. USA 89:space392-396 (1992); U.S. Pat. Nos. 5,270,184 and 5,455,166. Briefly, SDA uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (NASBA) described in U.S. Pat. No. 5,130,238; Q-beta replicase which uses an RNA replicase to amplify the probe molecule itself as described in Lizardi et al., BioTechnol. 6: 1197 (1988); transcription based amplification method described in Kwoh et al., Proc. Natl. Acad. Sci. USA 86:1173 (1989); and, self-sustained sequence replication described in Guatelli et al., Proc. Natl. Acad. Sci. USA 87: 1874 (1990). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified TMPRSS2/ERG transcript variants can be detected by any conventional means. For example, the TMPRSS2/ERG transcript variants can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. HPA is described in U.S. Pat. No. 5,283,174 and Norman C. Nelson et al., Nonisotopic Probing, Blotting, and Sequencing, ch. 17 (Larry J. Kricka ed., 2d ed. 1995).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205. Another method for determining the quantity of target sequence initially present in a sample, but which is not based on a real-time amplification, is disclosed in U.S. Pat. No. 5,710,029.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In one embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097.

Other self-hybridizing probes are well known to those of ordinary skill in the art. By way of non-limiting example, probe binding pairs having interacting labels, such as those disclosed in U.S. Pat. No. 5,928,862, might be adapted for use in the present invention. Probe systems used to detect single nucleotide polymorphisms (SNPs) might also be utilized in the present invention. Additional detection systems include “molecular switches,” as disclosed in U.S. Publ. No. 20050042638. Other probes, such as those comprising intercalating dyes and/or fluorochromes, are also useful for detection of amplification products in the present invention and are described in U.S. Pat. No. 5,814,447.

C. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

D. Compositions & Kits

Compositions for use in the diagnostic methods of the present invention include, but are not limited to, amplification oligonucleotides and probes. Any of these compositions, alone or in combination with other compositions of the present invention, may be provided in the form of a kit. For example, a pair of amplification oligonucleotides and a detection probe may be provided in a kit for the amplification and detection of the TMPRSS2/ERG transcript variants. Kits may further comprise appropriate controls and/or detection reagents.

The following examples are provided in order to demonstrate and further illustrate certain preferred embodiments and aspects of the present invention and are not to be construed as limiting the scope thereof.

### EXAMPLES

Amplification oligonucleotides and detection probes were designed, synthesized in vitro, and tested by making different combinations of amplification oligonucleotides (Table 1) in amplification reactions with synthetic target sequences and performing amplification reactions to determine the efficiency of amplification of the target sequences. The relative efficiencies of different combinations of amplification oligonucleotides were monitored by detecting the amplified products of the amplification reactions, generally by binding a labeled probe (Table 2) to the amplified products and detecting the relative amount of signal that indicated the amount of amplified product made.

Embodiments of amplification oligonucleotides for the 3′ UTR of TMPRSS2-ERG variants include those shown in Table 1. Amplification oligonucleotides include those that may function as primers, promoter-primers, and promoter-providers, with promoter sequences shown in lower case in Table 1. Some embodiments are the target-specific sequence of a promoter-primer or promoter-provider listed in Table 1, which optionally may be attached to the 3′ end of any known promoter sequence. An example of a promoter sequence specific for the RNA polymerase of bacteriophage T7 is SEQ ID NO: 46 (AATTTAATACGACTCACTATAGGGAGA). Embodiments of amplification oligonucleotides may include a mixture of DNA and RNA bases or 2′ methoxy linkages for the backbone joining RNA bases. Embodiments of amplification oligonucleotides may also be modified by synthesizing the oligonucleotide with a 3′ blocked to make them optimal for use in a single-primer transcription-mediated amplification reaction, i.e., functioning as blockers or promoter-providers. Preferred embodiments of 3′ blocked oligonucleotides include those of SEQ ID NOs: 16, 18, 20 and 22 that include a blocked C near or at the 3′ end.

TABLE 1

Amplification Oligonucleotides

| | |
|---|---|
| Sequence | SEQ ID |

| | |
|---|---|
| AGAGAAACATTCAGGACCTCATCATTATG | 2 |
| CAGGUCCTTCTTGCCTCCC | 3 |
| GCAGCCAAGAAGGCCATCT | 4 |
| aatttaatacgactcactatagggagaGCAGCCAAGAAGGCCATCT | 5 |
| TATGGAGGCTCCAATTGAAACC | 6 |
| aatttaatacgactcactatagggagaTATGGAGGCTCCAATTGAAACC | 7 |
| GGGCTGGTGAATGCACGCTGATGG | 12 |
| GUGGCGATGGGCTGGTGAATGCACGC | 13 |
| GAGUTTGTGGCGATGGGCTGGTGAATGC | 14 |
| CACCAACTGGGGGTATATACCCC | 15 |
| aatttaatacgactcactatagggagaccacaacggtttCACCAACTGGGGGTATATACCCC | 16 |
| GGGGGTATATACCCCAACACTAGGC | 17 |
| aatttaatacgactcactatagggagaccacaacggtttGGGGGTATATACCCCAACACTAGGC | 18 |
| GGTATATACCCCAACACTAGGCTCCCC | 19 |
| aatttaatacgactcactatagggagaccacaacggtttGGTATATACCCCAACACTAGGCTCCCC | 20 |
| CTCCCCACCAGCCATATGCCTTCTC | 21 |
| aatttaatacgactcactatagggagaccacaacggtttCTCCCCACCAGCCATATGCCTTCTC | 22 |

Embodiments of detection probes for amplified products of target sequences are shown in Table 2. Preferred detection probes form hairpin configurations by intramolecular hybridization of the probe sequence, which include those of SEQ ID NOs: 24, 26, 28 and 30 in Table 2, with the intramolecular hybridization sequences shown in lower case. Embodiments of hairpin probes were synthesized with a fluorescent label attached at one end of the sequence and a quencher compound attached at the other end of the sequence. Embodiments of hairpin probes may be labeled with a 5′ fluorophore and a 3′ quencher, for example a 5′ fluorescein label and a 3′ DABCYL quencher. Some embodiments of hairpin probes also include a non-nucleotide linker moiety at selected positions within the sequence. Examples of such embodiments include those that include an abasic 9-carbon (“C9”) linker between residues 5 and 6 of SEQ ID NO: 24, between residues 5 and 6 of SEQ ID NO: 26, between residues 19 and 20 of SEQ ID NO: 28, and between residues 24 and 25 of SEQ ID NO: 30.

TABLE 2
Detection Probes

| Sequence | SEQ ID |
|---|---|
| GCTTTGTTCTCCACAGGGTCAG | 8 |
| CTTTGTTCTCCACAGGGT | 9 |
| CTGTCTTTTATTTCTAGCCCCTTTTGG | 10 |
| GTCTTTTATTTCTAGCCCCTTTTGGAACAGG | 11 |
| UCUUUAGUAGUAAGUGCCCAG | 23 |
| cugggUCUUUAGUAGUAAGUGCCCAG | 24 |
| CCAGGUCUUUAGUAGUAAGUGCC | 25 |
| ggcucCCAGGUGUUUAGUAGUAAGUGCC | 26 |
| CCAGGUCUUUAGUAGUAAG | 27 |
| CCAGGUCUUUAGUAGUAAGccugg | 28 |
| CUCCGCCAGGUCUUUAGUAGUAAG | 29 |
| CUCCGCCAGGUCUUUAGUAGUAAGcggag | 30 |

Embodiments of target capture oligonucleotides for use in sample preparation to separate target nucleic acids from other sample components include those that contain the target-specific sequences in Table 3.

TABLE 3
Capture Oligonucleotides

| | SEQ |
|---|---|
| Sequence | ID |
| CUCCAUUACGCUGUGUCCUUUCUCC | 31 |
| CUUCCCCUUUCUCCAUUACGCUGUGUCC | 32 |
| GCGCAUUUUUGUUUCUGAAUUCUACUACUUCCCC | 33 |
| CATTTGACAAACAAAGAAAGAGATGCGC | 34 |
| CAGACAATTCCAGTTAAAATTTTCATTTG | 35 |
| CCAAACAUCCUAUUTCCUUGGCUCUCC | 36 |
| GAGAGGCUGACGCCAUUUGGGUGC | 37 |
| CCUAUUUCCUUGGCUCUCCCUUGC | 38 |
| UAACACUGGGUUUGGUAUAACACUG | 39 |
| CUGAAUUCUACUACUUCCCCUUU | 40 |
| GCGCAUUUUUGUUUCUGAAUUCUACUACUUCCCC | 41 |
| CAGACAAUUCCAGUUAAAAUUUUCAUUUGACAAACAAAGAAAGAG | 42 |
| CCGCCTACCCAAAATGCCTGCGTGATTTCTGATTG | 43 |
| CUGGAGGCCGCCUACCCAAAAUGCC | 44 |
| CGACUCAAAGGAAAACUGGAGGCCGCC | 45 |

Preferred embodiments of the capture oligonucleotides include a 3′ tail region covalently attached to the target-specific sequence to serve as a binding partner that binds a hybridization complex made up of the target nucleic acid and the capture oligonucleotide to an immobilized probe on a support. Preferred embodiments of capture oligonucleotides that include the target-specific sequences of SEQ ID NOs: 31-38 further include 3′ tail regions made up of substantially homopolymeric sequences, such as dT₃A₃₀ polymers.

Target capture may optionally include helper oligonucleotides that bind adjacent to target-specific capture oligonucleotides. The helper oligonucleotides are thought to aid in opening up the target nucleic acid thereby making it more accessible for capture. Preferred embodiments of helper oligonucleotides include SEQ ID NOs: 39-45.

Reagents used in target capture, amplification and detection steps in the examples described herein generally include one or more of the following. Sample Transport Solution contained 15 mM sodium phosphate monobasic, 15 mM sodium phosphate dibasic, 1 mM EDTA, 1 mM EGTA, and 3% (w/v) lithium lauryl sulfate, at pH 6.7. Urine Transport Medium contained 150 mM HEPES, 8% (w/v) lithium lauryl sulfate, 100 mM ammonium sulfate, and 2 M lithium hydroxide, and lithium hydroxide, monohydrate to pH 7.5. Target Capture Reagent contained 250 mM HEPES, 310 mM LiOH, 100 mM EDTA, 1800 mM LiCl, 0.250 mg/mL of paramagnetic particles (0.7-1.05μ particles, SERA-MAG™ MG-CM, Seradyn, Inc., Indianapolis, Ind.) with (dT)₁₄ oligonucleotides covalently bound thereto, and 0.01 μM target capture oligonucleotide. Wash Solution used in target capture contained 10 mM HEPES, 150 mM NaCl, 1 mM EDTA, 0.3% (w/v) ethyl alcohol, 0.02% (w/v) methyl paraben, 0.01% (w/v) propyl paraben and 0.1% (w/v) sodium dodecyl sulfate, at pH 7.5. Amplification reagent was a concentrated mixture that was mixed with other reaction components (e.g., sample or specimen dilution components) to produce a mixture containing 47.6 mM Na-HEPES, 12.5 mM N-acetyl-L-cysteine, 2.5% TRITON™ X-100, 54.8 mM KCl, 23 mM MgCl₂, 3 mM NaOH, 0.35 mM of each dNTP (dATP, dCTP, dGTP, dTTP), 7.06 mM rATP, 1.35 mM rCTP, 1.35 mM UTP, 8.85 mM rGTP, 0.26 mM Na₂EDTA, 5% (v/v) glycerol, 2.9% trehalose, 0.225% ethanol, 0.075% methylparaben, 0.015% propylparaben, and 0.002% Phenol Red, at pH 7.5-7.6. Amplification oligonucleotides (primers, promoter-primers, blockers, promoter-providers), and optionally probes, may be added to the reaction mixture in the amplification reagent or separate from the amplification reagent. Enzymes were added to TMA reaction mixtures at about 30 U/μL of MMLV reverse transcriptase (RT) and about 20 U/μL of T7 RNA polymerase per reaction (1 U of RT incorporates 1 nmol of dTTP in 10 min at 37° C. using 200-400 micromolar oligo dT-primed polyA template, and 1 U of T7 RNA polymerase incorporates 1 nmol of ATP into RNA in 1 hr at 37° C. using a T7 promoter in a DNA template). All of the reagent addition and mixing steps may be performed manually, using a combination of manual and automated steps, or by using a completely automated system. The amplification methods that use transcription-mediated amplification (TMA) substantially use the procedures already disclosed in detail in U.S. Pat. Nos. 5,399,491 and 5,554,516, Kacian et al. The amplification methods that use single-primer transcription-mediated amplification substantially use the procedures already disclosed in detail in US Pub. No. 2006-0046265. The methods for using hairpin probes are well-known, and include those already disclosed in detail in U.S. Pat. Nos. 6,849,412, 6,835,542, 6,534,274, and 6,361,945, Becker et al.

By using various combinations of these amplification oligonucleotides and detection probes, target sequences were specifically detected when the sample contained at least 15-50 copies of the target sequence. The following examples illustrate some of the embodiments of the invention for detection of target sequences.

### Example 1

### Transcription-Mediated Amplification and Detection

This example illustrates amplification and detection assays for target nucleic acid that detect amplified products at an end-point. The amplification reactions were transcription-mediated amplifications that used the procedures described in detail previously in U.S. Pat. Nos. 5,399,491 and 5,554,516, Kacian et al., using some of the amplification oligonucleotide embodiments described above. Synthetic target RNA of SEQ ID NO: 47 was captured using a target-specific capture oligonucleotide covalently bound to a dT₃A₃₀ polymer (5 pmol/reaction) in the presence of a helper oligonucleotide (5 pmol/reaction). Even though SEQ ID NO: 47 is given as DNA, the skilled artisan will appreciate that the corresponding RNA replaces all thymines (T) with uracil (U). Each of the assays was performed in an amplification reaction (0.075 mL total volume) that contained the target RNA and amplification reagents substantially as described above, with a promoter-primer (10 pmol/reaction) and a primer (15 pmol/reaction). The reaction mixtures containing the amplification oligonucleotides, target and amplification reagents (but not enzymes) were covered with 200 μL oil to prevent evaporation, incubated 10 min at 62° C., then 5 min at 42° C. After enzyme addition (25 μL), the reaction mixtures were mixed and incubated for 60 min at 42° C. Detection probe (0.05 pmol/reaction) was then added (100 μL) and hybridized by incubating for 20 min at 62° C. After 5 min at room temperature, selection reagent (250 μL) was added to cleave unhybridized detection probes during a 10 min incubation at 62° C. Once the reaction mixtures had cooled to room temperature, the RLU signals were measured 100 times at 0.02 second intervals in a HC+Leader.

TABLE 4
Calculated Signal:Noise Ratios Different Amplification
Oligonucleotide and Detection Probe Combinations
1 × 10⁵ Copies/Rxn Target

| | Signal:Noise Ratio | | | |
|---|---|---|---|---|
| Amplification | Detection Probes | | | |
| Oligonucleotide | SEQ ID | | SEQ ID | |
| Combinations | NO: 10 | SEQ ID NO: 11 | NO: 8 | SEQ ID NO: 9 |
| SEQ ID NOs: | 5 | 9 | NA | NA |
| 3 & 7 | | | | |
| SEQ ID NOs: | NA | NA | 1,305 | 988 |
| 2 & 5 | | | | |

NA = Not Applicable

These results indicate that SEQ ID NOs: 8 and 9 are suitable detection probes. SEQ ID NO: 8 was ultimately selected because of its higher signal:noise ratio and its significantly higher Tₘ. The calculated Tₘ of SEQ ID NO: 8 is 65.1° C. whereas the calculated Tₘ of SEQ ID NO: 9 is 60.9° C. Because hybridization of the detection probe to the target sequence is at 62° C., a detection probe with a Tₘ over 62° C. should perform more effectively.

TABLE 5
Measured Relative Light Units
Different Target Capture Oligonucleotides
Amplification Oligonucleotide SEQ ID NOs: 2 & 5
Detection Probe SEQ ID NO: 8

| | Relative Light Units (×1000) | | | | |
|---|---|---|---|---|---|
| Target | SEQ ID | SEQ ID | SEQ ID | SEQ ID | SEQ ID |
| (copies/rxn) | NO: 31 | NO: 32 | NO: 36 | NO: 37 | NO: 38 |
| 5,000 | 2,768 | 3,996 | 4,879 | 4,190 | 5,079 |
| 500 | 699 | 529 | 585 | 452 | 729 |
| 50 | 72 | 60 | 52 | 31 | 109 |

TABLE 6
Measured Relative Light Units
Different Target Capture Oligonucleotides
Amplification Oligonucleotide SEQ ID NOs: 2 & 5
Detection Probe SEQ ID NO: 8

Relative Light Units (×1000)

| Target | SEQ ID | SEQ ID | SEQ ID | SEQ ID |
|---|---|---|---|---|
| (copies/rxn) | NO: 31 | NO: 33 | NO: 34 | NO: 35 |
| 12,150 | 5,843 | 6,011 | 6,434 | 6,397 |
| 4,050 | 2,718 | 2,884 | 4,509 | 3,865 |
| 1,350 | 942 | 1,146 | 1,699 | 2,002 |
| 450 | 485 | 291 | 666 | 468 |
| 150 | 111 | 41 | 242 | 293 |
| 50 | 87* | 25 | 51 | 83 |
| 15 | 11 | 10 | 15 | 33 |

*1 of 5 replicates discarded as an outlier.

All of the capture oligonucleotides demonstrated at least 50 copies per reaction sensitivity. SEQ ID NO: 31 was ultimately selected because of its low standard deviations and linear output.

TABLE 7
Measured Relative Light Units
Different Helper Oligonucleotides
Target Capture Oligonucleotide SEQ ID NO: 31 or 34
Amplification Oligonucleotide SEQ ID NOs: 2 & 5
Detection Probe SEQ ID NO: 8

| | Relative Light Units (×1000) | | | | | | | |
|---|---|---|---|---|---|---|---|---|
| | SEQ ID NO: 31 | | | | SEQ ID NO: 34 | | | |
| Target | SEQ ID | SEQ ID | SEQ ID | SEQ ID | SEQ ID | SEQ ID | SEQ ID | SEQ ID |
| (copies/rxn) | NO: 39 | NO: 40 | NO: 41 | NO: 42 | NO: 41 | NO: 43 | NO: 44 | NO: 45 |
| 12,150 | 2,319 | 2,333 | 5,352 | NA | 7,552 | 7,352 | 7,224 | 7,204 |
| 4,050 | 739 | 924 | 1,816 | 6,926 | 7,504 | 7,394 | 7,205 | 7,234 |
| 1,350 | 257 | 265 | 727 | NA | 7,545 | 6,019 | 7,232 | 7,133 |
| 450 | 87 | 110 | 229 | 3,282 | 7,112 | 3,036 | 5,277 | 3,779 |
| 150 | 56 | 35 | 68 | 1,328 | 4,656 | 1,775 | 1,582 | 1,782 |
| 50 | 9 | 12 | 25 | 152 | 1,106 | 100 | 621 | 160 |
| 15 | 8 | 5 | 6 | 166 | 560 | 91 | 637 | 99 |

NA = Not Applicable

The results of these experiments demonstrate sensitivity from 15-50 copies per reaction.

Collectively, the results of the assays demonstrate a preferred combination of SEQ ID NOs: 31 and 41 for target capture and SEQ ID NOs: 2, 5 and 8 for transcription-mediated amplification and detection of the 3′ UTR of TMPRSS2-ERG variants.

### Example 2

### Single-Primer Transcription-Mediated Amplification and Detection

This example illustrates amplification and detection assays for target nucleic acid that detect amplified products in real-time. The amplification reactions were single-primer transcription-mediated amplifications that used the procedures described in detail previously in US Pub. No. 2006-0046265, using some of the amplification oligonucleotide embodiments described above. Each of the assays was performed in an amplification reaction (0.040 mL total volume) that contained synthetic target RNA of SEQ ID NO: 47 and amplification reagents substantially as described above, with a promoter-provider (6 pmol/reaction), a primer (6 pmol/reaction), a blocker (0.6 pmol/reaction), and a molecular torch (8 pmol/reaction). Embodiments of blockers include those shown in Table 8. The reaction mixtures containing the amplification oligonucleotides, target and amplification reagents (but not enzymes) were covered to prevent evaporation, incubated 10 min at 60° C., then 5 min at 42° C. Detection probes were added to the enzyme reagent at 0.8 pmol/μL. The resulting reagent was then added (10 μL) to the reaction mixtures and the reaction mixtures vortexed at 42° C. Fluorescence of the reaction mixtures was measured every 30 sec during the amplification reaction after enzyme addition.

TABLE 8
Blockers

| Sequence | SEQ ID |
|---|---|
| GGUGAAUUCCAGUAUGGGUUUGGGG | 48 |
| CCCCCAGUUGGUGAAUUCCAGUAUGGG | 49 |
| GGUAUAUACCCCCAGUUGGUGAAUUCC | 50 |
| GGGGUAUAUACCCCCAGUUGGUG | 51 |
| CCUAGUGUUGGGGUAUAUACCCCC | 52 |
| GGGGAGCCUAGUGUUGGG | 53 |

TABLE 9
Measured Time-of-Emergence
Different Promoter-Provider and Blocker Combinations
Primer SEQ ID NO: 14
Detection Probe SEQ ID NO: 30

Time-of-Emergence (min)

| | SEQ ID | SEQ ID | SEQ ID | SEQ ID |
|---|---|---|---|---|
| Target | NOs: | NOs: | NOs: | NOs: |
| (copies/rxn) | 16 & 50 | 18 & 50 | 20 & 51 | 22 & 53 |
| 0 | ND | ND | ND | ND |
| 50 | ND | 23.6 | 23.6 | ND |
| 10,000 | 37.7 | 17.1 | 15.3 | 23.1 |

ND = Not Detected

The results of this experiment demonstrate sensitivity at 50 copies per reaction with SEQ ID NO: 18 or 20 having much better performance than SEQ ID NO: 16 or 22.

TABLE 10
Measured Time-of-Emergence
Different Primers
Promoter-Provider and Blocker SEQ ID NOs: 20 & 51
Detection Probe SEQ ID NO: 30

| Target | Time-of-Emergence (min) | | |
|---|---|---|---|
| (copies/rxn) | SEQ ID NO: 12 | SEQ ID NO: 13 | SEQ ID NO: 14 |
| 0 | ND | ND | ND |
| 50 | 18.0 | 16.5 | 21.5 |
| 150 | 17.5 | 14.9 | 19.3 |
| 225 | 17.6 | 15.3 | 19.8 |
| 450 | 15.4 | 14.6 | 18.3 |
| 1350 | 15.4 | 15.4 | 17.0 |

ND = Not Detected

The results of this experiment demonstrate sensitivity at 50 copies per reaction with SEQ ID NO: 14 having much better linearity performance than SEQ ID NO: 12 or 13.

TABLE 11
Measured Time-of-Emergence
Different Detection Probes
Promoter-Provider and Blocker SEQ ID NOs: 20 & 51
Primer SEQ ID NO: 14

Time-of-Emergence (min)

| Target | SEQ ID | SEQ ID | SEQ ID | SEQ ID |
|---|---|---|---|---|
| (copies/rxn) | NO: 24 | NO: 26 | NO: 28 | NO: 30 |
| 0 | ND | ND* | ND | ND |
| 50 | 29.0 | 19.4 | 30.6 | 31.5 |
| 500 | 23.7 | 22.4 | 25.3 | 24.0 |
| 5000 | 20.2 | 18.3 | 21.0 | 19.9 |
| 50,000 | 17.7 | 15.6 | 18.4 | 17.2 |
| 500,000 | 15.9 | 13.7 | 16.5 | 15.2 |
| 5,000,000 | 12.8 | 11.0 | 13.4 | 12.3 |

ND = Not Detected
*1 of 3 replicates discarded as an outlier.

The measured average RFU ranges (RFUₘₐₓ−RFUₘᵢₙ) for SEQ ID NOs: 24, 26, 28 and 30 were 1.767, 0.628, 1.468 and 1.850, respectively. The results of the experiment demonstrate sensitivity at 50 copies per reaction with SEQ ID NO: 30 having much better performance and RFU dynamic range than SEQ ID NO: 24, 26 or 28.

TABLE 12
Measured Time-of-Emergence
Different Blockers
Primer and Promoter-Provider SEQ ID NOs: 14 & 20
Detection Probe SEQ ID NO: 30

| | Time-of-Emergence (min) | | | | | |
|---|---|---|---|---|---|---|
| Target | SEQ ID | SEQ ID | SEQ ID | SEQ ID | SEQ ID | SEQ ID |
| (copies/rxn) | NO: 48 | NO: 49 | NO: 50 | NO: 51 | NO: 52 | NO: 53 |
| 0 | ND | ND | ND | ND | ND | ND |
| 1,000 | 21.3 | 21.5 | 18.9 | 19.8 | 21.2 | 22.9 |
| 100,000 | 16.5 | 16.1 | 14.2 | 14.8 | 16.4 | 17.4 |

ND = Not Detected

TABLE 13
Measured Time-of-Emergence Standard Deviations
Different Blockers
Primer and Promoter-Provider SEQ ID NOs: 14 & 20
Detection Probe SEQ ID NO: 30

| | Standard Deviation (N = 4) | | | | | |
|---|---|---|---|---|---|---|
| Target | SEQ ID | SEQ ID | SEQ ID | SEQ ID | SEQ ID | SEQ ID |
| (copies/rxn) | NO: 48 | NO: 49 | NO: 50 | NO: 51 | NO: 52 | NO: 53 |
| 0 | ND | ND | ND | ND | ND | ND |
| 1,000 | 0.81 | 1.67 | 0.30 | 0.01 | 0.48 | 0.60 |
| 100,000 | 0.05 | 0.14 | 0.59 | 0.12 | 0.18 | 0.24 |

ND = Not Detected

The results of this experiment demonstrate sensitivity at 1,000 copies per reaction with SEQ ID NO: 51 having better performance and standard deviation than SEQ ID NO: 48, 49, 50, 52 or 53.

Collectively, the results of the assays demonstrate a preferred combination of SEQ ID NOs: 14, 18 or 20, 30 and 51 for single-primer transcription-mediated amplification and detection of the 3′ UTR of TMPRSS2-ERG variants.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method for amplifying and detecting an ERG nucleic acid in a sample comprising:
(a) contacting a biological sample containing an ERG nucleic acid with a first amplification oligonucleotide that specifically hybridizes to SEQ ID NO: 1 and a second amplification oligonucleotide that specifically hybridizes to SEQ ID NO: 1, wherein the first and second amplification oligonucleotides hybridize to different target sequences in SEQ ID NO: 1;
(b) exposing the sample contacted with the first amplification oligonucleotide and the second amplification oligonucleotide to conditions that amplify nucleic acids in vitro to make an amplified product from the ERG nucleic acid; and
(c) detecting the presence of the amplified product by specifically hybridizing the amplified product with a detection probe that specifically hybridizes to SEQ ID NO: 1 or a sequence completely complementary to SEQ ID NO: 1, thereby detecting the presence of the ERG nucleic acid in the biological sample.

2. The method of claim 1, further comprising a step before the contacting step to capture the ERG nucleic acid from the biological sample by specifically hybridizing the ERG nucleic acid with a capture olignucleotide selected from the group consisting of SEQ ID Nos. 31 to 38, and optionally hybridizing the ERG nucleic acid with a helper oligonucleotide selected from the group consisting of SEQ ID Nos. 39 to 45, to form a hybridization complex made up of at least the ERG nucleic acid hybridized to the capture olignucleotide, and then separating the hybridization complex from other components of the biological sample.

3. The method of claim 1, wherein the first amplification oligonucleotide is selected from the group consisting of SEQ ID Nos. 2 to 7 and 12 to 22, and wherein the second amplification oligonucleotide is selected from the group consisting of SEQ ID Nos. 2 to 7 and 12 to 22, provided that the first and second amplification oligonucleotides hybridize to different target sequences in SEQ ID NO: 1.

4. The method of claim 1, wherein the detecting step uses a detection probe selected from the group consisting of SEQ ID NOS. 8 to 11 and 23 to 30.

5. The method of claim 1, wherein the first amplification oligonucleotide consists of SEQ ID NO: 2 or SEQ ID NO:3 and the second amplification oligonucleotide consists of SEQ ID NO:4 or SEQ ID NO:4 covalently attached to a 5′ promoter sequence, or SEQ ID NO:6 or SEQ ID NO:6 covalently attached to a 5′ promoter sequence.

6. The method of claim 5, wherein the first amplification oligonucleotide consists of SEQ ID NO: 2, the second amplification oligonucleotide consists of SEQ ID NO:4 covalently attached to a 5′ promoter sequence, and the detection probe consists of SEQ ID NO:8 or SEQ ID NO:9.

7. The method of claim 5, wherein the first amplification oligonucleotide consists of SEQ ID NO: 3, the second amplification oligonucleotide consists of SEQ ID NO:6 covalently attached to a 5′ promoter sequence, and the detection probe consists of SEQ ID NO:10 or SEQ ID NO:11.

8. The method of claim 1, wherein the first amplification oligonucleotide is selected from the group consisting of SEQ ID Nos. 12, 13 and 14, and the second amplification oligonucleotide is selected from the group consisting of SEQ ID Nos. 15, SEQ ID NO:15 covalently attached to a 5′ promoter sequence, SEQ ID NO:16, SEQ ID NO:17 or SEQ ID NO:17 covalently attached to a 5′ promoter sequence, SEQ ID NO:18, SEQ ID NO:19 or SEQ ID NO:19 covalently attached to a 5′ promoter sequence, SEQ ID NO:20, SEQ ID NO:21 or SEQ ID NO:21 covalently attached to a 5′ promoter sequence, and SEQ ID NO:22.

9. The method of claim 8, wherein the contacting step further comprises contacting the ERG nucleic acid in the sample with a blocker oligonucleotide selected from SEQ ID Nos. 48 to 53.

10. The method of claim 8, wherein the detecting step uses a detection probe selected from the group consisting of SEQ ID NOS. 23 to 30.

11. The method of 10, wherein:
the first amplification oligonucleotide consists of SEQ ID NO:14, and the second amplification oligonucleotide consists of SEQ ID NO:16, SEQ ID NO:18, SEQ ID NO:20, or SEQ ID NO:22, the blocker oligonucleotide consists of SEQ ID NO:50, SEQ ID NO:51, or SEQ ID NO:53, and the detection probe consists of SEQ ID NO:30;
the first amplification oligonucleotide consists of SEQ ID NO:12, SEQ ID NO:13, or SEQ ID NO:14, and the second amplification oligonucleotide consists of SEQ ID NO:20, the blocker oligonucleotide consists of SEQ ID NO:51, and the detection probe consists of SEQ ID NO:30;
the first amplification oligonucleotide consists of SEQ ID NO:14, and the second amplification oligonucleotide consists of SEQ ID NO:20, the blocker oligonucleotide consists of SEQ ID NO:51, and the detection probe consists of SEQ ID NO:30;
the first amplification oligonucleotide consists of SEQ ID NO:14, and the second amplification oligonucleotide consists of SEQ ID NO:20, the blocker oligonucleotide consists of any one of SEQ ID Nos. 48 to 53, and the detection probe consists of SEQ ID NO:30; or
the first amplification oligonucleotide consists of SEQ ID NO:14, and the second amplification oligonucleotide consists of SEQ ID NO:18 or SEQ ID NO:20, the blacker oligonucleotide consists of SEQ ID NO:51, and the detection probe consists of SEQ ID NO:30.

12. A method for amplifying and detecting TMPRSS2/ERG transcript variants in a patient sample comprising:
(a) contacting the patient sample with a first amplification oligonucleotide comprising a target specific sequence consisting of SEQ ID NO: 14, a second amplification oligonucleotide comprising a target specific sequence consisting of SEQ ID NO: 17 or 19, and a detection probe comprising a target specific sequence consisting of SEQ ID NO: 29;
(b) exposing the patient sample to conditions of in vitro nucleic acid amplification sufficient to amplify TMPRSS2/ERG transcript variants by using the first and second amplification oligonucleotides to produce an amplified nucleic acid product; and
(c) detecting the amplified nucleic acid product by hybridizing the detection probe to the amplified nucleic acid product to detect the presence of TMPRSS2/ERG transcript variants in the patient sample.

13. A composition specific for detecting an ERG nucleic acid comprising:
a first amplification oligonucleotide comprising a sequence that specifically hybridizes to SEQ ID NO: 1;
a second amplification oligonucleotide comprising a sequence that specifically hybridizes to SEQ ID NO: 1; and
an oligonucleotide probe comprising a sequence that specifically hybridizes to SEQ ID NO: 1,
wherein the first amplification oligonucleotide is selected from the group consisting of SEQ ID Nos. 2 to 7 and 12 to 22, and wherein the second amplification oligonucleotide is selected from the group consisting of SEQ ID Nos. 2 to 7 and 12 to 22, provided that the first and second amplification oligonucleotides hybridize to different target sequences in SEQ ID NO: 1.

14. The composition of claim 13, further comprising a capture oligonucleotide that specifically hybridizes to SEQ ID NO: 47.

15. The composition of claim 14, wherein the capture oligonucleotide is selected from the group consisting of SEQ ID Nos. 31 to 38.

16. The composition of claim 15, further comprising a helper oligonucleotide selected from the group consisting of SEQ ID Nos. 39 to 45.

17. The composition of claim 13, wherein the oligonucleotide probe comprises a target specific sequence consisting of SEQ ID NO: 8, 9, 10, 11, 23, 25, 27 or 29.

18. The composition of claim 13, wherein the first amplification oligonucleotide consists of SEQ ID NO: 2 or SEQ ID NO:3, and the second amplification oligonucleotide consists of SEQ ID NO:4, SEQ ID NO:4 covalently attached to a 5′ promoter sequence, SEQ ID NO:5, SEQ ID NO:6, SEQ ID NO:6 covalently attached to a 5′ promoter sequence, or SEQ ID NO:7.

19. The composition of claim 13, wherein the first amplification oligonucleotide consists of SEQ ID Nos. 12, 13 or 14, and the second amplification oligonucleotide consists of SEQ ID NO:15, SEQ ID NO:15 covalently attached to a 5′ promoter sequence, SEQ ID NO:16, SEQ ID NO:17, SEQ ID NO:17 covalently attached to a 5′ promoter sequence, SEQ ID NO:18, SEQ ID NO:19, SEQ ID NO:19 covalently attached to a 5′ promoter sequence, SEQ ID NO:20, SEQ ID NO:21, SEQ ID NO:21 covalently attached to a 5′ promoter sequence, or SEQ ID NO:22.

20. The composition of claim 13, further comprising a blocker oligonucleotide selected from SEQ ID Nos. 48 to 53.
